(19) United States
(12) Patent Application Publication
(10) Pub. No.: US 2009/0318563 A1
(43) Pub. Date: Dec. 24, 2009

(54) Nematicidal Compositions and Methods

(75) Inventors: Michelle Coutu Hresko, Chesterfield, MO (US); Deryck J. Williams, S. Louis, MO (US); Merry B. McLaird, Kirkwood, MO (US); John D. Bradley, St. Louis, MO (US); Barry J. Shortt, New Melle, MO (US); Ronald E. Worthington, St. Louis, MO (US)

Correspondence Address: FISH & RICHARDSON PC, P.O. BOX 1022, MINNEAPOLIS, MN, 55440-1022, US

(73) Assignee: Divergence, Inc.

(21) Appl. No.: 12/342,720

(22) Filed: Dec. 23, 2008

Related U.S. Application Data
(63) Continuation of application No. 10/843,815, filed on May 12, 2004, now abandoned.
(60) Provisional application No. 60/470,061, filed on May 12, 2003.

(51) Int. Cl. A01N 33/08; C07C 215/06; C07C 215/20; A01P 5/00

(52) U.S. Cl. 514/653; 564/503; 564/355; 514/667

(57) ABSTRACT

Certain ethanolamine analogs and related compounds useful in the control of nematodes that infest plants or the situs of plants are described. Nematodes that parasitize animals can also be controlled using the methods and compounds of this invention

### CLAIM OF PRIORITY

This application is a continuation of U.S. application Ser. No. 10/843,815, filed May 12, 2004, which claims priority under 35 USC § 119(e) to U.S. Patent Application Ser. No. 60/470,061, filed on May 12, 2003, the entire contents of which are hereby incorporated by reference.

### BACKGROUND

Nematodes (derived from the Greek word for thread) are active, flexible, elongate, organisms that live on moist surfaces or in liquid environments, including films of water within soil and moist tissues within other organisms. While only 20,000 species of nematode have been identified, it is estimated that 40,000 to 10 million actually exist. Some species of nematodes have evolved to be very successful parasites of both plants and animals and are responsible for significant economic losses in agriculture and livestock and for morbidity and mortality in humans (Whitehead (1998) Plant Nematode Control. CAB International, New York).

Nematode parasites of plants can inhabit all parts of plants, including roots, developing flower buds, leaves, and stems. Plant parasites are classified on the basis of their feeding habits into the broad categories: migratory ectoparasites, migratory endoparasites, and sedentary endoparasites. Sedentary endoparasites, which include the root knot nematodes (Meloidogyne) and cyst nematodes (Globodera and Heterodera) induce feeding sites and establish long-term infections within roots that are often very damaging to crops (Whitehead, supra). It is estimated that parasitic nematodes cost the horticulture and agriculture industries in excess of $78 billion worldwide a year, based on an estimated average 12% annual loss spread across all major crops. For example, it is estimated that nematodes cause soybean losses of approximately $3.2 billion annually worldwide (Barker et al. (1994) Plant and Soil Nematodes: Societal Impact and Focus for the Future. The Committee on National Needs and Priorities in Nematology. Cooperative State Research Service, US Department of Agriculture and Society of Nematologists). Several factors make the need for safe and effective nematode controls urgent. Continuing population growth, famines, and environmental degradation have heightened concern for the sustainability of agriculture, and new government regulations may prevent or severely restrict the use of many available agricultural anthelmintic agents.

There are a very small array of chemicals available to control nematodes (Becker (1999) Agricultural Research Magazine 47(3):22-24; U.S. Pat. Nos. 6,048,714). Nevertheless, the application of chemical nematicides remains the major means of nematode control. In general, chemical nematicides are highly toxic compounds known to cause substantial environmental damage and are increasingly restricted in the amounts and locations in which they can be used. For example, the soil fumigant methyl bromide which has been used effectively to reduce nematode infestations in a variety of specialty crops, is regulated under the U.N. Montreal Protocol as an ozone-depleting substance and is scheduled for elimination in 2005 in the US (Carter (2001) California Agriculture, 55(3):2). It is expected that strawberry and other commodity crop industries will be significantly impacted if a suitable replacement for methyl bromide is not found. Similarly, broad-spectrum nematicides such as Telone (various formulations of 1,3-dichloropropene) have significant restrictions on their use because of toxicological concerns (Carter (2001) California Agriculture, Vol. 55(3):12-18).

The macrocyclic lactones (e.g., avermectins and milbemycins) and delta-toxins from Bacillus thuringiensis (Bt) are chemicals that in principle provide excellent specificity and efficacy and should allow environmentally safe control of plant parasitic nematodes. Unfortunately, in practice, these two nematicidal agents have proven less effective in agricultural applications against root pathogens. Although certain avermectins show exquisite activity against plant parasitic nematodes, these chemicals are hampered by poor bioavailability due to their light sensitivity, degradation by soil microorganisms and tight binding to soil particles (Lasota & Dybas (1990) Acta Leiden 59(1-2):217-225; Wright & Perry (1998) Musculature and Neurobiology. In: The Physiology and Biochemistry of Free-Living and Plant-parasitic Nematodes (eds R. N. Perry & D. J. Wright), CAB International 1998). Consequently despite years of research and extensive use against animal parasitic nematodes, mites and insects (plant and animal applications), macrocyclic lactones (e.g., avermectins and milbemycins) have never been commercially developed to control plant parasitic nematodes in the soil.

Bt delta toxins must be ingested to affect their target organ, the brush border of midgut epithelial cells (Marroquin et al. (2000) Genetics. 155(4):1693-1699). Consequently they are not anticipated to be effective against the dispersal, non-feeding, juvenile stages of plant parasitic nematodes in the field. Because juvenile stages only commence feeding when a susceptible host has been infected, nematicides may need to penetrate the plant cuticle to be effective. Transcuticular uptake of a 65-130 kDa protein—the size of typical Bt delta toxins—is unlikely. Furthermore, soil mobility is expected to be relatively poor. Even transgenic approaches are hampered by the size of Bt delta toxins because delivery in planta is likely to be constrained by the exclusion of large particles by the feeding tubes of certain plant parasitic nematodes such as Heterodera (Atkinson et al. (1998) Engineering resistance to plant-parasitic nematodes. In: The Physiology and Biochemistry of Free-Living and Plant-parasitic Nematodes (eds R. N. Perry & D. J. Wright), CAB International 1998).

Fatty acids are a class of natural compounds that have been investigated as alternatives to the toxic, non-specific organophosphate, carbamate and fumigant pesticides (Stadler et al. (1994) Planta Medica 60(2):128-132; U.S. Pat. Nos. 5,192,546; 5,346,698; 5,674,897; 5,698,592; 6,124,359). It has been suggested that fatty acids derive their pesticidal effects by adversely interfering with the nematode cuticle or hypodermis via a detergent (solubilization) effect, or through direct interaction of the fatty acids and the lipophilic regions of target plasma membranes (Davis et al. (1997) Journal of Nematology 29(4S):677-684). In view of this predicted mode of action it is not surprising that fatty acids are used in a variety of pesticidal applications including as herbicides (e.g., SCYTHE by Dow Agrosciences is the C9 saturated fatty acid pelargonic acid), bactericides and fungicides (U.S. Pat. Nos. 4,771,571; 5,246,716) and insecticides (e.g., SAFER INSECTICIDAL SOAP by Safer, Inc.).

The phytotoxicity of fatty acids has been a major constraint on their general use in post-plant agricultural applications (U.S. Pat. No. 5,093,124) and the mitigation of these undesirable effects while preserving pesticidal activity is a major area of research. Post-plant applications are desirable because of the relatively short half-life of fatty acids under field conditions.

The esterification of fatty acids can significantly decrease their phytotoxicity (U.S. Pat. Nos. 5,674,897; 5,698,592; 6,124,359). Such modifications can however lead to loss of nematicidal activity as is seen for linoleic, linolenic and oleic acid (Stadler et al. (1994) Planta Medica 60(2):128-132) and it may be impossible to completely decouple the phytotoxicity and nematicidal activity of pesticidal fatty acids because of their non-specific mode of action. Perhaps not surprisingly, the nematicidal fatty acid pelargonic acid methyl ester (U.S. Pat. Nos. 5,674,897; 5,698,592; 6,124,359) shows a relatively small “therapeutic window” between the onset of pesticidal activity and the observation of significant phytotoxicity (Davis et al. (1997) J Nematol 29(4S):677-684). This is the expected result if both the phytotoxicity and the nematicidial activity derive from the non-specific disruption of plasma membrane integrity.

Ricinoleic acid, the major component of castor oil, has been shown to have an inhibitory effect on water and electrolyte absorption using everted hamster jejunal and ileal segments (Gaginella et al. (1975) J Pharmacol Exp Ther 195(2):355-61) and to be cytotoxic to isolated intestinal epithelial cells (Gaginella et al. (1977) J Pharmacol Exp Ther 201(1):259-66). These features are likely the source of the laxative properties of castor oil which is given as a purgative in humans and livestock (e.g., castor oil is a component of some de-worming protocols because of its laxative properties). In contrast, the methyl ester of ricinoleic acid is ineffective at suppressing water absorption in the hamster model (Gaginella et al. (1975) J Pharmacol Exp Ther 195(2):355-61).

Many plant species are known to be highly resistant to nematodes. The best documented of these include marigolds (Tagetes spp.), rattlebox (Crotalaria spectabilis), chrysanthemums (Chrysanthemum spp.), castor bean (Ricinus communis), margosa (Azardiracta indica), and many members of the family Asteraceae (family Compositae) (Hackney & Dickerson. (1975) J Nematol 7(1):84-90). In the case of the Asteraceae, the photodynamic compound alpha-terthienyl has been shown to account for the strong nematicidal activity of the roots. Castor beans are plowed under as a green manure before a seed crop is set. However, a significant drawback of the castor plant is that the seed contains toxic compounds (such as ricin) that can kill humans, pets, and livestock and is also highly allergenic. In many cases however, the active principle(s) for plant nematicidal activity has not been discovered and it remains difficult to derive commercially successful nematicidal products from these resistant plants or to transfer the resistance to crops of agronomical importance such as soybeans and cotton.

Genetic resistance to certain nematodes is available in some commercial cultivars (e.g., soybeans), but these are restricted in number and the availability of cultivars with both desirable agronomic features and resistance is limited. The production of nematode resistant commercial varieties by conventional plant breeding based on genetic recombination through sexual crosses is a slow process and is often further hampered by a lack of appropriate germplasm.

There remains an urgent need to develop environmentally safe, target-specific ways of controlling plant parasitic nematodes. In the specialty crop markets, economic hardship resulting from nematode infestation is highest in strawberries, bananas, and other high value vegetables and fruits. In the high-acreage crop markets, nematode damage is greatest in soybeans and cotton. There are however, dozens of additional crops that suffer from nematode infestation including potato, pepper, onion, citrus, coffee, sugarcane, greenhouse ornamentals and golf course turf grasses.

Nematode parasites of vertebrates (e.g., humans, livestock and companion animals) include gut roundworms, hookworms, pinworms, whipworms, and filarial worms. They can be transmitted in a variety of ways, including by water contamination, skin penetration, biting insects, or by ingestion of contaminated food.

In domesticated animals, nematode control or “de-worming” is essential to the economic viability of livestock producers and is a necessary part of veterinary care of companion animals. Parasitic nematodes cause mortality in animals (e.g., heartworm in dogs and cats) and morbidity as a result of the parasites' inhibiting the ability of the infected animal to absorb nutrients. The parasite-induced nutrient deficiency leads to disease and stunted growth in livestock and companion animals. For instance, in cattle and dairy herds, a single untreated infection with the brown stomach worm can permanently restrict an animal's ability to convert feed into muscle mass or milk.

Two factors contribute to the need for novel anthelmintics and vaccines to control animal parasitic nematodes. First, some of the more prevalent species of parasitic nematodes of livestock are building resistance to the anthelmintic drugs available currently, meaning that these products will eventually lose their efficacy. These developments are not surprising because few effective anthelmintic drugs are available and most have been used continuously. Some parasitic species have developed resistance to most of the anthelmintics (Geents et al. (1997) Parasitology Today 13:149-151; Prichard (1994) Veterinary Parasitology 54:259-268). The fact that many of the anthelmintic drugs have similar modes of action complicates matters, as the loss of sensitivity of the parasite to one drug is often accompanied by side resistance—that is, resistance to other drugs in the same class (Sangster & Gill (1999) Parasitology Today 15(4):141-146). Secondly, there are some issues with toxicity for the major compounds currently available.

Infections by parasitic nematode worms result in substantial human mortality and morbidity, especially in tropical regions of Africa, Asia, and the Americas. The World Health Organization estimates 2.9 billion people are infected, and in some areas, 85% of the population carries worms. While mortality is rare in proportion to infections, morbidity is substantial and rivals diabetes and lung cancer in worldwide disability adjusted life year (DALY) measurements.

Examples of human parasitic nematodes include hookworms, filarial worms, and pinworms. Hookworms (1.3 billion infections) are the major cause of anemia in millions of children, resulting in growth retardation and impaired cognitive development. Filarial worms invade the lymphatics, resulting in permanently swollen and deformed limbs (elephantiasis), and the eyes, causing African river blindness. The large gut roundworm Ascaris lumbricoides infects more than one billion people worldwide and causes malnutrition and obstructive bowel disease. In developed countries, pinworms are common and often transmitted through children in daycare.

Even in asymptomatic parasitic infections, nematodes can still deprive the host of valuable nutrients and increase the ability of other organisms to establish secondary infections. In some cases, infections can cause debilitating illnesses and can result in anemia, diarrhea, dehydration, loss of appetite, or death.

Despite some advances in drug availability and public health infrastructure and the near elimination of one tropical nematode (the water-borne Guinea worm), most nematode diseases have remained intractable problems. Treatment of hookworm diseases with anthelmintic drugs, for instance, has not provided adequate control in regions of high incidence because rapid re-infection occurs after treatment. In fact, over the last 50 years, while nematode infection rates have fallen in the United States, Europe, and Japan, the overall number of infections worldwide has kept pace with the growing world population. Large scale initiatives by regional governments, the World Health Organization, foundations, and pharmaceutical companies are now underway attempting to control nematode infections with currently available tools, including three programs for control of Onchocerciasis (river blindness) in Africa and the Americas using ivermectin and vector control; The Global Alliance to Eliminate Lymphatic Filariasis using DEC, albendazole, and ivermectin; and the highly successful Guinea Worm Eradication Program. Until safe and effective vaccines are discovered to prevent parasitic nematode infections, anthelmintic drugs will continue to be used to control and treat nematode parasitic infections in both humans and domestic animals.

Finding effective compounds and vaccines against parasitic nematodes has been complicated by the fact that the parasites have not been amenable to culturing in the laboratory. Parasitic nematodes are often obligate parasites (i.e., they can only survive in their respective hosts, such as in plants, animals, and/or humans) with slow generation times. Thus, they are difficult to grow under artificial conditions, making genetic and molecular experimentation difficult or impossible. To circumvent these limitations, scientists have used Caenorhabidits elegans as a model system for parasitic nematode discovery efforts.

C. elegans is a small free-living bacteriovorous nematode that for many years has served as an important model system for multicellular animals (Burglin (1998) Int. J. Parasitol. 28(3):395-411). The genome of C. elegans has been completely sequenced and the nematode shares many general developmental and basic cellular processes with vertebrates (Ruvkin et al. (1998) Science 282:2033-41). This, together with its short generation time and ease of culturing, has made it a model system of choice for higher eukaryotes (Aboobaker et al. (2000) Ann. Med. 32:23-30).

Although C. elegans serves as a good model system for vertebrates, it is an even better model for study of parasitic nematodes, as C. elegans and other nematodes share unique biological processes not found in vertebrates. For example, unlike vertebrates, nematodes produce and use chitin, have gap junctions comprised of innexin rather than connexin and contain glutamate-gated chloride channels rather than glycine-gated chloride channels (Bargmann (1998) Science 282:2028-33). The latter property is of particular relevance given that the avermectin class of drugs is thought to act at glutamate-gated chloride receptors and is highly selective for invertebrates (Martin (1997) Vet. J. 154:11-34).

A subset of the genes involved in nematode-specific processes will be conserved in nematodes and absent or significantly diverged from homologues in other phyla. In other words, it is expected that at least some of the genes associated with functions unique to nematodes will have restricted phylogenetic distributions. The completion of the C. elegans genome project and the growing database of expressed sequence tags (ESTs) from numerous nematodes facilitate identification of these “nematode-specific” genes. In addition, conserved genes involved in nematode-specific processes are expected to retain the same or very similar functions in different nematodes. This functional equivalence has been demonstrated in some cases by transforming C. elegans with homologous genes from other nematodes (Kwa et al. (1995) J. Mol. Biol. 246:500-10; Redmond et al. (2001) Mol. Biochem. Parasitol. 112:125-131). This sort of data transfer has been shown in cross phyla comparisons for conserved genes and is expected to be more robust among species within a phylum. Consequently, C. elegans and other free-living nematode species are likely excellent surrogates for parasitic nematodes with respect to conserved nematode processes.

Many expressed genes in C. elegans and certain genes in other free-living nematodes can be “knocked out” genetically by a process referred to as RNA interference (RNAi), a technique that provides a powerful experimental tool for the study of gene function in nematodes (Fire et al. (1998) Nature 391(6669):806-811; Montgomery et al. (1998) Proc. Natl. Acad Sci USA 95(26):15502-15507). Treatment of a nematode with double-stranded RNA of a selected gene can destroy expressed sequences corresponding to the selected gene thus reducing expression of the corresponding protein. By preventing the translation of specific proteins, their functional significance and contribution to the fitness of the nematode can be assessed. Determination of essential genes and their corresponding proteins using C. elegans as a model system will assist in the rational design of anti-parasitic nematode control products.

The present invention describes compositions which show surprising nematicidal activity in part due to selective inhibition of metabolic processes demonstrated to be essential to nematodes and either absent or non-essential in vertebrates and plants. This invention therefore provides urgently needed compounds and methods for the environmentally safe control of parasitic nematodes.

### SUMMARY

The invention concerns compositions and processes for controlling nematodes. In one embodiment, the subject invention comprises the use of certain compounds, including ethanolamine analogs and related compounds to control nematodes that infest plants or the situs of plants. Nematodes that parasitize animals can also be controlled using the methods and compounds of this invention.

Certain of the useful nematicidal ethanolamine analogs are predicted inhibitors of nematode phosphoethanolamine N-methyltransferase and related enzymes (also referred to herein as nematode PEAMT enzymes). These useful ethanolamine analogs can be, for example, alcohols, phosphates, phosphonic acids, sulfonic acids, sulfonamides, sulfonyl fluorides, trifluoromethyl sulfones and trifluoromethyl sulfonamides, phosphate esters, phosphonate esters and sulfonate esters which can be activated to the corresponding acid forms in vivo. The compounds can also contain a substituant, e.g., a halogen, in place of hydrogen at certain positions. In certain embodiments, the ethanolamine analogs are PEAMT inhibiting phosphate diesters, phosphonate diesters or sulfonate esters which can be activated to the corresponding phosphate, phosphonate and sulfonate analogs in vivo. In the sulfonate ester, phosphonate diester or phosphate diester the ionizable protons are replaced with other functional groups (e.g., phenyl or alkyl groups) in order to improve cell membrane permeability.

Useful ethanolamine analogs include N-substituted ethanolamine analogs such as 2-(diisopropylamino)ethanol, 2-(tert-butylamino)ethanol and N-(2-hydroxyethyl)aniline and C-substituted ethanolamine analogs such as D-phenylalaninol. Useful compounds also include N- or C-substituted derivatives of phosphoethanolamine (phosphate analogs), derivatives of 2-aminoethylphosphonic acid and 3-aminopropylphosphonic acid (phosphonate analogs), and taurine derivatives (sulfonate analogs). Examples of such ethanolamine analogs are 2-amino-3-phenylpropyl phosphonic acid (phosphonate analog) and N-phenyltaurine (sulfonate analog). Among the useful compounds are sulfonate esters, phosphonate diesters and phosphate diesters such as alkyl, phenyl or alkoxyalkyl esters which can be activated to the corresponding sulfonic acid, phosphate or phosphonate compound in vivo. Other useful analogs have non-ionizable groups in place of the phosphate moiety. Such compounds include alkyl compounds (e.g., N-ethylaniline, 4-(N-ethyl-N-methylamino)azobenzene), sulfonyl fluorides (e.g., 2-(4-phenylazo-phenylamino)-ethanesulfonyl fluoride, 2-[4-(4-dimethylamino-phenylazo)-phenylamino]-ethanesulfonyl fluoride), sulfonamides (e.g., 2-(4-phenylazo-phenylamino)-ethanesulfonamide, 2-[4-(4-dimethylamino-phenylazo)-phenylamino]-ethanesulfonamide), trifluoromethyl sulfonamides (e.g., C,C,C-trifluoro-N-(2-phenylamino-ethyl)-methanesulfonamide) and trifluoromethyl sulfones. Certain methylene (CH₂) carbons (e.g., phosphonate) may or may not have their hydrogens substituted, e.g., with fluorine (e.g., fluorinated phosphonate).

Specifically excluded from this invention are the natural substrates or products of ethanolamine methyltransferases and phosphoethanolamine N-methyltransferases such as ethanolamine (EA) or phosphoethanolamine (pEA), monomethylethanolamine (MME) or phosphomonomethylethanolamine (pMME), dimethylethanolamine (DME) or phosphodimethylethanolamine (pDME), choline (Cho) or phosphocholine (pCho) and their corresponding phosphate esters.

Ethanolamine analogs (e.g., alcohols, phosphates, phosphonates, fluorophosphonates sulfonates, sulfonyl fluorides, sulfonamides, trifluoromethyl sulfonamides, trifluoromethyl sulfones, phosphate diesters, phosphonate diesters and sulfonate esters) that have the characteristics of a specific inhibitor of a PEAMT inhibit the activity of a nematode phosphoethanolamine N-methyltransferase to a lesser extent in the presence of products of the methyltransferase reaction (e.g., MME, pMME, DME, pDME, Cho, pCho) than in the presence of substrates of the enzyme (e.g., EA, pEA, MME, pMME, DME, pDME). For these competition experiments the substrate (e.g., pEA) and the product (e.g., pMME) are used in equivalent amounts. In competition experiments, uncharged precursors to the phosphorylated chemicals such as EA and MME capable of in vivo conversion to the corresponding phosphobases (e.g., pEA or pMME) can also be used. These effects can be demonstrated on a phosphoethanolamine N-methyltransferase (also referred to herein as a PEAMT) protein in vitro, on transgenic cells containing PEAMTs or on intact organisms (e.g., a nematode) containing PEAMT. In one embodiment of this test, the inhibitor, the substrate (or uncharged substrate precursor) and product (or uncharged product precursor) of the PEAMT are present in equal concentrations.

The invention also features compounds that inhibit the expression of a PEAMT at the level of transcription or translation. Also within the invention are compounds that impair the modification of a PEAMT resulting in a change in the activity or localization of the methyltransferase.

The invention also features compounds that are relatively selective inhibitors of one or more nematode PEAMT polypeptides relative to one or more plant or animal PEAMT-like polypeptides or phosphatidylethanolamine N-methyltransferase polypeptides. The compounds can have a Kᵢ for a nematode PEAMT that is 10-fold, 100-fold, 1,000-fold or more lower than for plant or animal methyltransferase-like polypeptides, e.g., a host plant or host animal of the nematode. The invention further features relatively non-selective inhibitors as well as completely non-selective inhibitors.

In yet another aspect, the invention features a method of treating a disorder (e.g., an infection) caused by a nematode, (e.g., M. incognita, H. glycines, H. contortus, A. suum) in a subject, e.g., a host plant, animal, or person. The method includes administering to the subject an effective amount of a compound of the invention, e.g., an inhibitor of a PEAMT polypeptide activity or an inhibitor of expression of a PEAMT polypeptide or an inhibitor that impairs the modification of a PEAMT resulting in change in the activity or localization of the methyltransferase. The inhibitor may be delivered by several means including pre-planting, post-planting and as a feed additive, drench, external application, pill or by injection.

In still another aspect, methods of inhibiting a nematode (e.g., M. incognita, H. glycines, H. contortus, A. suum) PEAMT(s) are provided. Such methods can include the steps of: (a) providing a nematode that contains a PEAMT-like gene; (b) contacting the nematode with an ethanolamine analog (alcohol, phosphate, phosphonate, sulfonate, phosphate diester, phosphonate diester and/or sulfonate ester) or other compounds that inhibit the enzyme. Also provided are methods of rescuing the effect of the inhibitor. Such methods comprise the steps of: (a) inhibiting the enzyme and (b) providing PEAMT products or product precursors exogenously (e.g., dimethylethanolamine or choline).

In another aspect, methods of reducing the viability or fecundity or slowing the growth or development or inhibiting the infectivity of a nematode using a nematicidal ethanolamine analog of the invention, e.g., an inhibitor of a PEAMT are provided. Such methods comprise the steps of (a) providing a nematode that contains a PEAMT-like gene; (b) contacting the nematode with specific ethanolamine analogs, e.g., an inhibitor of a PEAMT; (c) reducing the viability or fecundity of the nematode. Also provided are methods of rescuing the effect of the methyltransferase inhibitors or other inhibitors. Such methods can involve contacting the nematode exogenously with ethanolamine or phosphoethanolamine methylation products or product precursors (e.g., MME, pMME, DME, pDME, Cho, pCho).

The invention features a method for reducing the viability, growth, or fecundity of a nematode, the method comprising exposing the nematode to an ethanolamine analog of the invention, e.g., a compound that inhibits the activity of a PEAMT-like polypeptide (e.g., a PEAMT) and a method of protecting a plant from a nematode infection, the method comprising applying to the plant, to the soil, or to seeds of the plant an ethanolamine analog of the invention.

The invention also features a method for protecting a vertebrate (e.g., a bird or a mammal) from a nematode infection, the method comprising administering to the vertebrate an ethanolamine analog of the invention, e.g., an inhibitor of a nematode PEAMT-like polypeptide (e.g., a PEAMT enzyme). In preferred embodiments the inhibitor does not significantly inhibit the activity of a PEAMT-like polypeptide or phosphatidylethanolamine N-methyltransferase-like polypeptide expressed by the vertebrates or at least does not do so to the extent that the growth of the vertebrate is significantly impaired. The bird can be a domesticated fowl (e.g., a chicken, turkey, duck, or goose). The mammal can be a domesticated animal, e.g., a companion animal (e.g., a cat, dog, horse or rabbit) or livestock (e.g., a cow, sheep, pig, goat, alpaca or llama).

The invention process is particularly valuable to control nematodes attacking the roots of desired crop plants, ornamental plants, and turf grasses. The desired crop plants can be, for example, soybeans, cotton, corn, tobacco, wheat, strawberries, tomatoes, banana, sugar cane, sugar beet, potatoes, or citrus.

Thus, the invention features a composition, e.g., a nematicidal composition, comprising: an effective amount of a compound or a mixture of compounds having the formula:

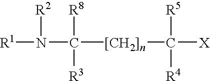

wherein:

each R¹, R², R³ and R⁸ is, independently, singly or multiply substituted or unsubstituted alkyl, cycloalkyl, alkenyl, alkynyl, or aromatic group that can be an aryl or heteroaryl group (e.g., aryl, phenylazophenyl, pyrrolidine, benzotriazol, benzoimidazol, imidazole, indole, purine, pyrimidine groups); or a hydroxy, alkoxy, oxo, or hydrogen;

each R⁴ and R⁵ is, independently, hydrogen or halogen;

n=0 or 1;

X=—H, an optionally independently substituted alkyl group, —OH —OPO₃H₂ (phosphate), —PO₃H₂ (phosphonate), or —SO₃H (sulfonate), or —SO₂F (sulfonyl fluoride), —SO₂NH₂ (sulfonamide), —NHSO₂CF₃ (trifluoromethyl sulfonamide), —SO₂CF₃ (trifluoromethyl sulfone), —OPO₃R⁶R⁷ (phosphate ester or diester), —PO₃R⁶R⁷ (phosphonate ester or diester), or —SO₃R⁶ (sulfonate ester);

R⁶=unsubstituted, singly substituted or multiply substituted alkyl (e.g., C1-C6 or C1-C3), phenyl, alkoxyalkyl or alkoxyaryl;

R⁷=unsubstituted, singly substituted or multiply substituted alkyl (e.g., C1-C6 or C1-C3), phenyl, alkoxyalkyl or alkoxyaryl.

In certain embodiments, R¹ or R² (but not both R¹ and R²) is an unsubstituted, singly or multiply substituted alkyl group (e.g., C1-C6 or C1-C3 alkyl such as tert-butyl or isopropyl) or an unsubstituted, singly or multiply substituted aryl group (e.g., phenyl or phenylazophenyl). In other embodiments both R¹ and R² are unsubstituted, singly or multiply substituted alkyl groups (e.g., C1-C6 or C1-C3 alkyl such as tert-butyl, isopropyl) or unsubstituted, singly or multiply substituted aryl groups (e.g., phenyl). In other embodiments R³ (but not R¹ or R²) is an unsubstituted, singly or multiply substituted alkyl groups (e.g., tert-butyl, isopropyl) or an unsubstituted, singly or multiply substituted aryl groups (e.g., phenyl or benzyl). R¹ and R² in certain embodiments are C₁-C₅ (e.g., C₃-C₄) alkyl groups.

R¹, R², R³ and R⁸ groups include substituted or unsubstituted straight or branched C₁-C₁₂ alkyl (e.g., C₁-C₁₀, C₁-C₈, C₁-C₆, C₁-C₄, C₁-C₃); substituted or unsubstituted straight or branched; C₂-C₁₂ alkenyl (e.g., C₂-C₁₀, C₂-C₈, C₂-C₆, C₂-C₄); substituted or unsubstituted straight or branched C₂-C₁₂ alkynyl (e.g., C₂-C₁₀, C₂-C₈, C₂-C₆, C₂-C₄); C₃-C₈ (e.g., C₃-C₇, C₃-C₆, C₃-C₅) cycloalkyl; and C₆-C₁₀ aryl. Preferred substituents for R³ include benzyl, C₃-C₈ cycloalkyl, halo, hydroxy, mercapto, C₁-C₁₀ alkoxy, C₁-C₁₀ thioalkoxy, amino, C₁-C₁₀ alkylamino, C₁-C₁₀ dialkylamino, C₁-C₁₀ haloalkyl, acyl and oxo.

In some embodiments n=0, R⁴ and R⁵=H, and X is an OH or phosphate or phosphate diester. In some of these embodiments X is a phosphate diester with R⁵ and/or R⁶ comprising unsubstituted, singly substituted or multiply substituted alkyl, phenyl, alkoxyalkyl or alkoxyphenyl groups. In other embodiments R³ and R⁸=H. In other embodiments n=0 or 1, R⁴ and R⁵=F, or R⁴ and R⁵=H, and X is a hydrogen or an alkyl group or phosphonate or sulfonate or sulfonyl fluoride or sulfonamide or trifluoromethyl sulfonamide or trifluoromethyl sulfone or phosphonate diester or sulfonate ester. In some of these embodiments X is a phosphonate diester or sulfonate ester with R⁶ and/or R⁷ comprising unsubstituted, singly substituted or multiply substituted alkyl, phenyl, alkoxyalkyl or alkoxyphenyl groups.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by a halogen, and includes alkyl moieties in which all hydrogens have been replaced by a halogen (e.g., perfluoroalkyl). The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 9-fluorenyl, benzhydryl, and trityl groups.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent.

The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “alkoxy” refers to an —O-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl. Bicyclic aryl groups can have, e.g., 10 ring carbon atoms.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl, cycloalkyl, haloalkyl (e.g., perfluoroalkyl), aryl, heteroaryl, aralkyl, heteroaralkyl, heterocyclyl, alkenyl, alkynyl, cycloalkenyl, heterocycloalkenyl, alkoxy, haloalkoxy (perfluoroalkoxy), halo, hydroxy, carboxy, carboxylate, cyano, nitro, amino, alkyl aminosulfonate, sulfonate, sulfate, phosphate, methylenedioxy, ethylenedioxy, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The compositions can also include one or more nematicides such as an avermectin (e.g., ivermectin), milbemycin, aldicarb, oxamyl, fenamiphos, fosthiazate or metam sodium. The composition may also include insecticides (e.g., cinnamaldehyde, sucrose octaonate esters, spinosad), herbicides (e.g., trifloxysulfuron, glyphosate, halosulfuron) and other chemicals for disease control (e.g., chitosan). The nematicidal compositions can also comprise co-solvents, permeation enhancers and aqueous surfactants.

A permeation enhancer is generally an agent that facilitates the active compounds of the invention, e.g., the ethanolamine analogs of the invention, to pass through cellular membranes.

A co-solvent (i.e., a latent solvent or indirect solvent) is an agent that becomes an effective solvent in the presence of an active solvent and can improve the properties of the primary (active) solvent.

The composition can be produced in concentrated form that includes little or no water. The composition can be diluted with water or some other solvent prior to use to treat plants, seeds, soil or vertebrates.

The invention also features a nematicidal composition comprising: ethanolamine analogs or mixture of analogs selected from the group consisting of alkyl compounds N-ethylaniline and 4-(N-ethyl-N-methylamino)azobenzene, sulfonyl fluorides 2-(4-phenylazo-phenylamino)-ethanesulfonyl fluoride and 2-[4-(4-dimethylamino-phenylazo)-phenylamino]-ethanesulfonyl fluoride, sulfonamides 2-(4-phenylazo-phenylamino)-ethanesulfonamide and 2-[4-(4-dimethylamino-phenylazo)-phenylamino]-ethanesulfonamide, the trifluoromethyl sulfonamide C,C,C-Trifluoro-N-(2-phenylamino-ethyl)-methanesulfonamide, alcohols 2-(diisopropylamino)ethanol, 2-(tert-butylamino)ethanol, N-(2-hydroxyethyl)aniline and D-phenylalaninol and their phosphate, phosphate diester, phosphonate, phosphonate diester, alpha-fluorinated phosphonate, alpha-fluorinated phosphonate diester, sulfonate and sulfonate esters. Preferred esters include methyl esters, ethyl esters, phenyl esters, alkoxyalkyl (e.g., pivaloyloxymethyl) esters and alkoxyphenyl (e.g., phenoxyethyl) esters.

In various preferred embodiments the composition further comprises an aqueous surfactant or surfactant mixture selected from the group consisting of: ethyl lactate, Span 20, Span 40, Span 80, Span 85, Tween 20, Tween 40, Tween 80, Tween 85, Triton X 100, Makon 10, Igepal CO 630, Brij 35, Brij 97, Tergitol TMN 6, Dowfax 3B2, Physan and Toximul TA 15; the composition further comprises a permeation enhancer (e.g., cyclodextrin); the composition further comprises a co-solvent (e.g., isopropanol, acetone, 1,2-propanediol, a petroleum based-oil (e.g., aromatic 200) or a mineral oil (e.g., paraffin oil)); the composition further comprises a nematicide selected from the group consisting of: avermectins (e.g., ivermectin), milbemycin, aldicarb, oxamyl, fenamiphos, fosthiazate and metam sodium. The composition may also comprise insecticides (e.g., cinnamaldehyde, sucrose octaonate esters, spinosad), herbicides (e.g., trifloxysulfuron, glyphosate, halosulfuron) and other chemicals for disease control (e.g., chitosan).

The invention features methods for controlling nematodes by administering an ethanolamine analog or mixture ethanolamine analogs of the invention, e.g., a PEAMT inhibitor. Thus, the invention includes a method for control of unwanted nematodes, the method comprising administering to vertebrates, plants, seeds or soil a nematicidal composition comprising: (a) an effective amount of a compound or a mixture of compounds having the formula:

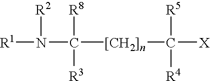

wherein:

each R¹, R², R³ and R⁸ is, independently, singly or multiply substituted or unsubstituted alkyl, cycloalkyl, alkenyl, alkynyl, or aromatic group that can be an aryl or heteroaryl group (e.g., aryl, phenylazophenyl, pyrrolidine, benzotriazol, benzoimidazol, imidazole, indole, purine, pyrimidine groups); or a hydroxy, alkoxy, oxo, or hydrogen;

each R⁴ and R⁵ is, independently, hydrogen or halogen;

n=0 or 1;

X=—H, an optionally independently substituted alkyl group, —OH—OPO₃H₂ (phosphate), —PO₃H₂ (phosphonate), or —SO₃H (sulfonate), or —SO₂F (sulfonyl fluoride), —SO₂NH₂ (sulfonamide), —NHSO₂CF₃ (trifluoromethyl sulfonamide), —SO₂CF₃ (trifluoromethyl sulfone), —OPO₃R⁶R⁷ (phosphate ester or diester), —PO₃R⁶R⁷ (phosphonate ester or diester), or —SO₃R⁶ (sulfonate ester);

R⁶=unsubstituted, singly substituted or multiply substituted alkyl (e.g., C1-C6 or C1-C3), phenyl, alkoxyalkyl or alkoxyaryl;

R⁷=unsubstituted, singly substituted or multiply substituted alkyl (e.g., C1-C6 or C1-C3), phenyl, alkoxyalkyl or alkoxyaryl.

In certain embodiments, R¹ or R² (but not both R¹ and R²) is an unsubstituted, singly or multiply substituted alkyl group (e.g., C1-C6 or C1-C3 alkyl such as tert-butyl or isopropyl) or an unsubstituted, singly or multiply substituted aryl group (e.g., phenyl or phenylazophenyl). In other embodiments both R¹ and R² are unsubstituted, singly or multiply substituted alkyl groups (e.g., C1-C6 or C1-C3 alkyl such as tert-butyl, isopropyl) or unsubstituted, singly or multiply substituted aryl groups (e.g., phenyl). In other embodiments R³ (but not R¹ or R²) is an unsubstituted, singly or multiply substituted alkyl groups (e.g., tert-butyl, isopropyl) or an unsubstituted, singly or multiply substituted aryl groups (e.g., phenyl or benzyl). R¹ and R² in certain embodiments are C₁-C₅ (e.g., C₃-C₄) alkyl groups.

R¹, R², R³ and R⁸ groups include substituted or unsubstituted straight or branched C₁-C₁₂ alkyl (e.g., C₁-C₁₀, C₁-C₈, C₁-C₆, C₁-C₄, C₁-C₃); substituted or unsubstituted straight or branched; C₂-C₁₂ alkenyl (e.g., C₂-C₁₀, C₂-C₈, C₂-C₆, C₂-C₄); substituted or unsubstituted straight or branched C₂-C₁₂ alkynyl (e.g., C₂-C₁₀, C₂-C₈, C₂-C₆, C₂-C₄); C₃-C₈ (e.g., C₃-C₇, C₃-C₆, C₃-C₅) cycloalkyl; and C₆-C₁₀ aryl. Preferred substituents for R³ include benzyl, C₃-C₈ cycloalkyl, halo, hydroxy, mercapto, C₁-C₁₀ alkoxy, C₁-C₁₀ thioalkoxy, amino, C₁-C₁₀ alkylamino, C₁-C₁₀ dialkylamino, C₁-C₁₀ haloalkyl, acyl and oxo.

In some embodiments n=0, R⁴ and R⁵=H, and X is an OH or phosphate or phosphate diester. In some of these embodiments X is a phosphate diester with R⁵ and/or R⁶ comprising unsubstituted, singly substituted or multiply substituted alkyl, phenyl, alkoxyalkyl or alkoxyphenyl groups. In other embodiments R³ and R⁸=H. In other embodiments n=0 or 1, R⁴ and R⁵=F, or R⁴ and R⁵=H, and X is a hydrogen or an alkyl group or phosphonate or sulfonate or sulfonyl fluoride or sulfonamide or trifluoromethyl sulfonamide or trifluoromethyl sulfone or phosphonate diester or sulfonate ester. In some of these embodiments X is a phosphonate diester or sulfonate ester with R⁶ and/or R⁷ comprising unsubstituted, singly substituted or multiply substituted alkyl, phenyl, alkoxyalkyl or alkoxyphenyl groups.

The compositions can also include one or more nematicides such as an avermectin (e.g., ivermectin), milbemycin, aldicarb, oxamyl, fenamiphos, fosthiazate or metam sodium. The composition may also include insecticides (e.g., cinnamaldehyde, sucrose octaonate esters, spinosad), herbicides (e.g., trifloxysulfuron, glyphosate, halosulfuron) and other chemicals for disease control (e.g., chitosan). The nematicidal compositions can also comprise co-solvents, permeation enchancers and aqueous surfactants.

The invention also features a method for control of unwanted nematodes comprising administering to vertebrates, plants, seeds or soil a nematicidal composition comprising an effective amount of: (a) ethanolamine analog or a mixture of ethanolamine analogs selected from the group consisting of alkyl compounds N-ethylaniline and 4-(N-ethyl-N-methylamino)azobenzene, sulfonyl fluorides 2-(4-phenylazo-phenylamino)-ethanesulfonyl fluoride and 2-[4-(4-dimethylamino-phenylazo)-phenylamino]-ethanesulfonyl fluoride, sulfonamides 2-(4-phenylazo-phenylamino)-ethanesulfonamide and 2-[4-(4-dimethylamino-phenylazo)-phenylamino]-ethanesulfonamide, the trifluoromethyl sulfonamide C,C,C-Trifluoro-N-(2-phenylamino-ethyl)-methanesulfonamide), alcohols 2-(diisopropylamino)ethanol, 2-(tert-butylamino)ethanol and N-(2-hydroxyethyl)aniline and D-phenylalaninol and their phosphate, phosphate diester, phosphonate, phosphonate diester, fluorophosphonate, alpha-fluorinated phosphonate diester, sulfonate and sulfonate esters. Preferred esters include methyl esters, ethyl esters, phenyl esters, alkoxyalkyl (e.g., pivaloyloxymethyl) esters and alkoxyphenyl (e.g., phenoxyethyl) esters.

In certain embodiments of the method the composition further comprises an aqueous surfactant or surfactant mixture selected from the group consisting of: ethyl lactate, Span 20, Span 40, Span 80, Span 85, Tween 20, Tween 40, Tween 80, Tween 85, Triton X 100, Makon 10, Igepal CO 630, Brij 35, Brij 97, Tergitol TMN 6, Dowfax 3B2, Physan and Toximul TA 15; the composition may comprise a permeation enhancer (e.g., a cyclodextrin); the composition may comprise a co-solvent (e.g., isopropanol, acetone, 1,2-propanediol, a petroleum based-oil (e.g., aromatic 200) or a mineral oil (e.g., paraffin oil)); the method includes administering (before, after or in conjunction with the ethanolamine analog) a nematicide selected from the group consisting of avermectins (e.g., ivermectin), milbemycin, aldicarb, oxamyl, fenamiphos, fosthiazate and metam sodium, an insecticide (e.g., cinnamaldehyde, sucrose octaonate esters, spinosad), a herbicide (e.g., trifloxysulfuron, glyphosate, halosulfuron) and/or other chemicals for disease control (e.g., chitosan); the nematode infects plants and the nematicidal composition is applied to the soil or to plants; the nematicidal composition is applied to soil before planting; the nematicidal composition is applied to soil after planting; the nematicidal composition is applied to soil using a drip system; the nematicidal composition is applied to soil using a drench system; the nematicidal composition is applied to plant roots; the nematicidal composition is applied to seeds; the nematicidal composition is applied to the foliage of plants; the nematode infects a vertebrate; the nematicidal composition is administered to a bird or non-human mammal; the nematicidal composition is administered to a human; the nematicidal composition is formulated as a drench to be administered to a non-human animal; the nematicidal composition is formulated as an orally administered drug; and the nematicidal composition is formulated as an injectable drug.

The invention also features feeds that have been supplemented to include one or more of the compounds of the invention, e.g., a phosphoethanolamine N-methyltransferase inhibitor. The feeds may also be treated to reduce the amount of a phosphoethanolamine N-methyltransferase substrates or products in the feed. More generally, the feed can be treated to reduce the content of choline that could act to complement the loss of PEAMT activity.

Thus, the invention features a nematicidal feed for a non-human vertebrate comprising: (a) an animal feed; (b) an effective amount of a nematicidal compound or mixtures of compounds having the formula:

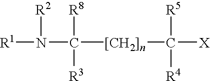

wherein:

each R¹, R², R³ and R⁸ is, independently, singly or multiply substituted or unsubstituted alkyl, cycloalkyl, alkenyl, alkynyl, or aromatic group that can be an aryl or heteroaryl group (e.g., aryl, phenylazophenyl, pyrrolidine, benzotriazol, benzoimidazol, imidazole, indole, purine, pyrimidine groups); or a hydroxy, alkoxy, oxo, or hydrogen;

each R⁴ and R⁵ is, independently, hydrogen or halogen;

n=0 or 1;

X=—H, an optionally independently substituted alkyl group, —OH—OPO₃H₂ (phosphate), —PO₃H₂ (phosphonate), or —SO₃H (sulfonate), or —SO₂F (sulfonyl fluoride), —SO₂NH₂ (sulfonamide), —NHSO₂CF₃ (trifluoromethyl sulfonamide), —SO₂CF₃ (trifluoromethyl sulfone), —OPO₃R⁶R⁷ (phosphate ester or diester), —PO₃R⁶R⁷ (phosphonate ester or diester), or —SO₃R⁶ (sulfonate ester);

R⁶=unsubstituted, singly substituted or multiply substituted alkyl (e.g., C1-C6 or C1-C3), phenyl, alkoxyalkyl or alkoxyaryl;

R⁷=unsubstituted, singly substituted or multiply substituted alkyl (e.g., C1-C6 or C1-C3), phenyl, alkoxyalkyl or alkoxyaryl.

In certain embodiments, R¹ or R² (but not both R¹ and R²) is an unsubstituted, singly or multiply substituted alkyl group (e.g., C1-C6 or C1-C3 alkyl such as tert-butyl or isopropyl) or an unsubstituted, singly or multiply substituted aryl group (e.g., phenyl or phenylazophenyl). In other embodiments both R¹ and R² are unsubstituted, singly or multiply substituted alkyl groups (e.g., C1-C6 or C1-C3 alkyl such as tert-butyl, isopropyl) or unsubstituted, singly or multiply substituted aryl groups (e.g., phenyl). In other embodiments R³ (but not R¹ or R²) is an unsubstituted, singly or multiply substituted alkyl groups (e.g., tert-butyl, isopropyl) or an unsubstituted, singly or multiply substituted aryl groups (e.g., phenyl or benzyl). R¹ and R² in certain embodiments are C₁-C₅ (e.g., C₃-C₄) alkyl groups.

R¹, R², R³ and R⁸ groups include substituted or unsubstituted straight or branched C₁-C₁₂ alkyl (e.g., C₁-C₁₀, C₁-C₈, C₁-C₆, C₁-C₄, C₁-C₃); substituted or unsubstituted straight or branched; C₂-C₁₂ alkenyl (e.g., C₂-C₁₀, C₂-C₈, C₂-C₆, C₂-C₄); substituted or unsubstituted straight or branched C₂-C₁₂ alkynyl (e.g., C₂-C₁₀, C₂-C₈, C₂-C₆, C₂-C₄); C₃-C₈ (e.g., C₃-C₇, C₃-C₆, C₃-C₅) cycloalkyl; and C₆-C₁₀ aryl. Preferred substituents for R³ include benzyl, C₃-C₈ cycloalkyl, halo, hydroxy, mercapto, C₁-C₁₀ alkoxy, C₁-C₁₀ thioalkoxy, amino, C₁-C₁₀ alkylamino, C₁-C₁₀ dialkylamino, C₁-C₁₀ haloalkyl, acyl and oxo.

In some embodiments n=0, R⁴ and R⁵=H, and X is an OH or phosphate or phosphate diester. In some of these embodiments X is a phosphate diester with R⁵ and/or R⁶ comprising unsubstituted, singly substituted or multiply substituted alkyl, phenyl, alkoxyalkyl or alkoxyphenyl groups. In other embodiments R³ and R⁸=H. In other embodiments n=0 or 1, R⁴ and R⁵=F, or R⁴ and R⁵=H, and X is a hydrogen or an alkyl group or phosphonate or sulfonate or sulfonyl fluoride or sulfonamide or trifluoromethyl sulfonamide or trifluoromethyl sulfone or phosphonate diester or sulfonate ester. In some of these embodiments X is a phosphonate diester or sulfonate ester with R⁶ and/or R⁷ comprising unsubstituted, singly substituted or multiply substituted alkyl, phenyl, alkoxyalkyl or alkoxyphenyl groups.

The feed can be treated to reduce choline content. The feed can be selected from the group consisting of: soy, wheat, corn, sorghum, millet, alfalfa, clover, and rye.

As used herein, an agent with “anthelmintic or anthelminthic or antihelminthic activity” is an agent, which when tested, has measurable nematode-killing activity or results in reduced fertility or sterility in the nematodes such that fewer viable or no offspring result, or compromises the ability of the nematode to infect or reproduce in its host, or interferes with the growth or development of a nematode. The agent may also display nematode repellant properties. In the assay, the agent is combined with nematodes, e.g., in a well of microtiter dish, in liquid or solid media or in the soil containing the agent. Staged nematodes are placed on the media. The time of survival, viability of offspring, and/or the movement of the nematodes are measured. An agent with “anthelmintic or anthelminthic or antihelminthic activity” can, for example, reduce the survival time of adult nematodes relative to unexposed similarly staged adults, e.g., by about 20%, 40%, 60%, 80%, or more. In the alternative, an agent with “anthelmintic or anthelminthic or antihelminthic activity” may also cause the nematodes to cease replicating, regenerating, and/or producing viable progeny, e.g., by about 20%, 40%, 60%, 80%, or more. The effect may be apparent immediately or in successive generations.

As used herein, the term “altering an activity” refers to a change in level, either an increase or a decrease in the activity, (e.g., an increase or decrease in the ability of the polypeptide to bind or regulate other polypeptides or molecules) particularly a PEAMT-like activity (e.g., the ability to methylate pEA, pMME or pDME). The change can be detected in a qualitative or quantitative observation. If a quantitative observation is made, and if a comprehensive analysis is performed over a plurality of observations, one skilled in the art can apply routine statistical analysis to identify modulations where a level is changed and where the statistical parameter, the p value, is, for example, less than 0.05.

In part, the nematicidal ethanolamine analogs described herein provide an effective, environmentally safe means of inhibiting nematode metabolism, growth, viability, fecundity, development, infectivity and/or the nematode life-cycle. The compounds may be used alone or in combination with other nematicidal agents.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a set of drawings depicting the structures of ethanolamine and its methylated analogs monomethylethanolamine (MME), dimethylethanolamine (DME) and choline chloride (Cho Cl). Also shown are phosphoethanolamine (pEA) a substrate of PEAMTs, two phosphonic analogs of pEA (2-aminoethylphosphonic acid and 3-aminopropylphosphonic acid) and a sulfonic analog of pEA (taurine).

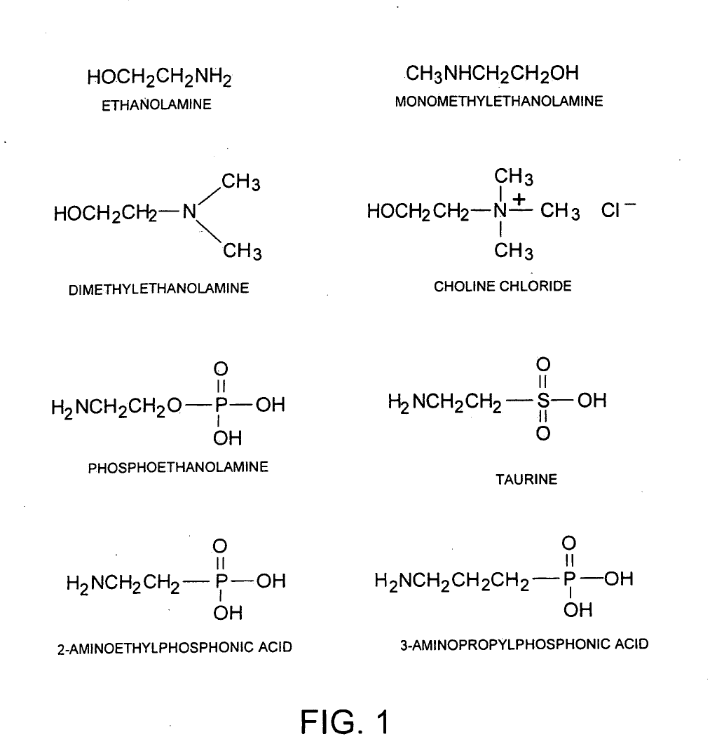

FIG. 2 depicts drawings of four nematicidal ethanolamine (alcohol) analogs: 2-(diisopropylamino)ethanol, 2-(tert-butlylamino)ethanol, D-phenylalaninol and N-(2-hydroxyethyl)aniline.

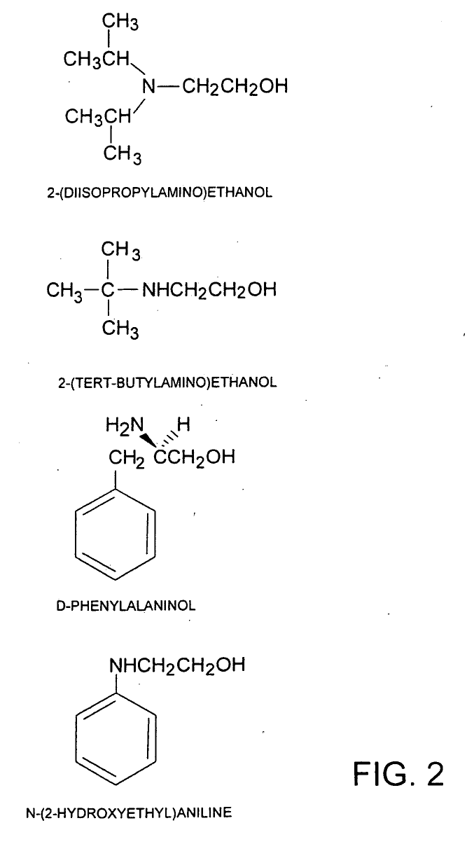

FIG. 3 shows ethanolamine and a sulfonic acid analog taurine and the nematicidal N-(2-hydroxyethyl)aniline analog and its corresponding sulfonic acid analog N-phenyltaurine.

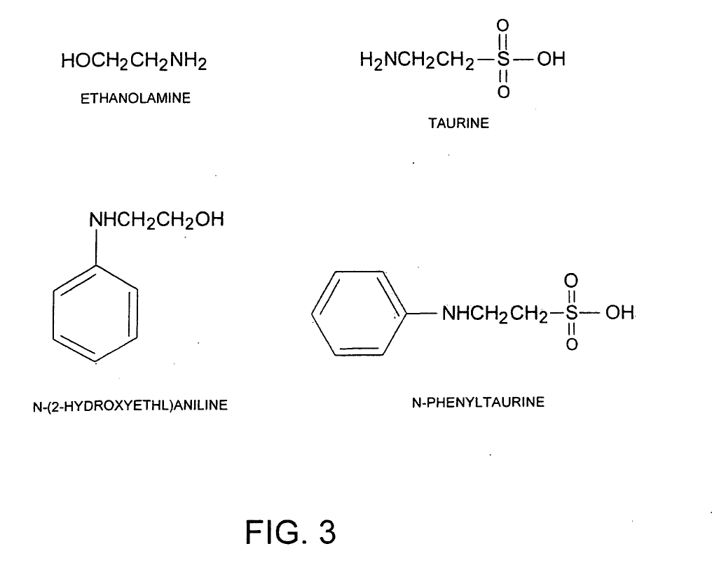

FIG. 4 shows a test of 2-(4-phenylazo-phenylamino)-ethanesulfonyl fluoride (3746) against root knot nematode (Meloidogyne incognita) on tomato plants grown in pots. Active ingredients are added to the soil to mimic three field rates of 25, 10 and 5 kilograms per hectare. Top panel shows the degree of nematode control (gall ratings) and lower panel the assessment of phytotoxicity (root weights)

### DETAILED DESCRIPTION

Choline (Cho) plays a number of important roles in biological systems. In bacteria, fungi, plants and animals, phosphatidylcholine is a major component of membrane phospholipids and the free base is a precursor to the neurotransmitter acetylcholine in animals. Choline is also an intermediate in glycine betaine (a compound that increases tolerance to osmotic stresses) synthesis in plants (McNeil et al. (2001) Proc Natl Acad Sci USA 98: 10001-5). Choline is an essential nutrient in humans and other animals, and also plays a critical role in brain development in humans (Sheard et al. (1986) Am J Clin Nutr. 1986 43:219-24; Tayek et al. (1990) J Am Coll Nut 9:76-83). Most organisms can incorporate choline into phosphatidylcholine using a pathway that transfers a choline moiety from CDP-choline to diacylglycerol. In similar fashion, choline precursors such as ethanolamine (EA), monomethylethanolamine (MME) and dimethylethanolamine (DME) can also be incorporated into phospholipids via the CPD-choline or Kennedy pathway. Rhizobacteria have an additional Kennedy-independent pathway that also allows the incorporation of choline excreted from plant roots directly into phospholipids (Rudder et al. (1999) J Biol Chem. 274:20011-6; Lopez-Lara & Geiger (2001) J Biotechnol 91:211-21).

Among those organisms that can synthesize choline, different biosynthetic pathways are used to make choline from ethanolamine via the successive addition of methyl groups using S-adenosyl methionine (SAM) as the methyl donor. These pathways differ in whether they use the free base (ethanolamine), the phosphobase (phosphoethanolamine), or the phosphatidyl base (phosphatidylethanolamine) as the methylation substrate. Plants are unusual in that they can methylate the free base, phosphobase or phosphatidylbase (phospholipid substrate) (Bolognese & McGraw (2000) Plant Physiol. 124(4):1800-13; Nuccio et al. (2000) J Biol Chem 275(19):14095-101; Charron et al. (2002). Plant Physiol. 129(1):363-73). However, the conversion of phosphatidylethanolamine to phosphatidylmonomethylethanolamine has not been demonstrated in plants, so the first methylation reaction probably occurs at either the free base or the phosphobase level. It is now thought that in many plants the major flux occurs at the phosphobase level, catalyzed by the phosphoethanolamine N-methyltransferase enzyme (PEAMT) (i.e., pEA [symbol] pMME).

In contrast, in most other organisms, methylation is carried out primarily at the phospholipid level. The complete reaction (i.e., Ptd-EA [symbol] Ptd-MME [symbol] Ptd-DME [symbol] PtdCho) requires a single enzyme in bacteria and mammals and two separate enzymes in fungi (Kanipes & Henry. (1997) Biochim Biophys Acta. 1348(1-2):134-41; Vance et al. (1997) Biochim Biophys Acta. 1348(1-2):142-50; Hanada et al. (2001) Biosci Biotechnol Biochem. 65(12):2741-8). Mammalian nerve cells are reported to have additional phopho-base methylation activity and three distinct enzymes appear to be involved (Andriamampandry et al. (1992) Biochem J. 288 (1):267-72; Mukherjee et al. (1995) Neurochem Res. 20(10):1233-7).

Plant methyltransferases from spinach and Arabidopsis have been cloned by complementation of choline biosynthetic mutants in fission and budding yeast, respectively (Bolognese & McGraw (2000) Plant Physiol. 124(4):1800-13; Nuccio et al. (2000) J Biol. Chem. 275(19):14095-101). In contrast to yeast methyltransferases, which act on the phosphatidylethanolamine, these plant enzymes have been shown to act on phosphoethanolamine. A similar gene has recently been cloned from chilled wheat tissues (Charron et al. (2002). Plant Physiol. 129(1):363-73). The plant enzymes are predicted to encode soluble proteins of approximately 55 kDa that have two domains containing separate SAM binding sites. Each domain contains motifs—termed I, post-I, II, and III—that are conserved among SAM-dependent methyltransferases. cDNA clones encompassing partial sequence from both SAM binding sites have been isolated from numerous plants, including Oryza sativa, Brassica napus, Gossypium hirsutum, and Hordeum vulgare. The plant methyltransferase structure is thought to have arisen from a gene duplication event, since prokaryotic and animal methyltransferases are approximately half the size of the plant enzymes and have only one methyltransferase domain.

Some basic kinetic characteristics of the spinach methyltransferase have been determined from enzyme preparations isolated from fission yeast overexpressing it. Enzyme activity is dependent on SAM and phosphoethanolamine concentrations. In the presence of these substrates, methyltransferase-containing extracts catalyze the formation of monomethyl- and dimethylphosphoethanolamine as well as phosphocholine. The appearance of these intermediates suggests that they are precursors to phosphocholine. A truncated version of the spinach enzyme lacking the second SAM binding site can accomplish the first methylation converting phosphoethanolamine to monomethylphosphoethanolamine, but cannot perform the second and third methylation steps. It is presumed that the C-terminal half can carry out the second and third methylation reactions.

The C. elegans genome contains two PEAMT-like genes and several homologs are found in other nematode EST datasets suggesting that these genes are widely distributed in Nematoda. The nematode proteins and plant homologs are all presumably localized in the cytosol as in the case of the wheat PEAMT as they lack secretion leaders (analyzed by methods at www.cbs.dtu.dk/services/TargetP) or transmembrane regions (analyzed by methods at www.cbs.dtu.dk/services/TMHMM). One of the C. elegans PEAMT genes (PEAMT2) encodes a polypeptide which is 437 amino acids long (accession number AAB04824.1, wormbase locus F54D11.1) and shows significant similarity to the C-terminal half of the spinach PEAMT and other plant homologs with two SAM binding domains. The second C. elegans PEAMT gene appears to encode at least to two different splice variants (PEAMT1a and PEAMT1b). PEAMT1a and b are 495 and 484 amino acids long, respectively (accession number AAA81102.1, wormbase locus ZK622.3a and ZK622.3b) and are most similar to the N-terminal half of the plant PEAMTs. A PFAM analysis (at www.pfam.wust1.edu) supports the blast predictions that whereas the plant PEAMTs contain two canonical methyltransferase domains, the nematode proteins contain an N-terminal MT domain in PEAMT1 and a C-terminal MT domain in PEAMT2. PEAMT1 and PEAMT2 have 30-40% amino acid identity to their plant homologs in the regions that align. The similarity between PEAMT1 and PEAMT2 is low (22% amino acid identity) and is restricted to a small 127 amino acid region in their C-terminal domains.

Given the similarity of PEAMT1 and PEAMT2 to the N- and C-terminal domains of the plant PEAMTs (e.g. spinach and Arabidopsis) respectively, their similar larval lethal RNAi phenotypes and the observation that the N-terminal half of the spinach enzyme is only capable of the first methylation reaction, we predicted that PEAMT1 would catalyze the conversion of pEA to pMME (the first methylation) and PEAMT2 would catalyze the conversion of pMME to pDME and pDME to pCHO. This hypothesis was confirmed by chemical complementation of the C. elegans PEAMT1 or PEAMT2 RNAi phenotypes with EA, MME, DME or Cho (see Table 1). As predicted, the PEAMT1 larval lethal RNAi phenotype is suppressed by MME, DME and Cho but not by EA whereas the PEAMT2 RNAi is rescued only by Cho and not by MME, DME, or EA singly or in combination.

We have further made the surprising discovery that certain N-substituted and C-substituted ethanolamine analogs (e.g., N-ethylaniline, 4-(N-ethyl-N-methylamino)azobenzene, 2-(4-phenylazo-phenylamino)-ethanesulfonyl fluoride, 2-[4-(4-dimethylamino-phenylazo)-phenylamino]-ethanesulfonyl fluoride, 2-(4-phenylazo-phenylamino)-ethanesulfonamide, 2-[4-(4-dimethylamino-phenylazo)-phenylamino]-ethanesulfonamide, C,C,C-Trifluoro-N-(2-phenylamino-ethyl)-methanesulfonamide, 2-(diisopropylamino)ethanol, 2-(tert-butylamino)ethanol, N-(2-hydroxyethyl)aniline and D-phenylalaninol; see Tables 3, 4 and 5) are nematicidal and have activity consistent with that of specific inhibitors of nematode PEAMTs. These ethanolamine analogs and their phosphate diesters, phosphonate diesters, fluorinated phosphonate diesters and sulfonate esters can be used effectively to control parasitic nematodes while minimizing undesirable damage to non-target organisms.

Ethanolamine analogs or other types of PEAMT inhibitors may be supplied to plants exogenously, through sprays for example. These inhibitory analogs may also be applied as a seed coat. It is also possible to provide inhibitors through a host organism or an organism on which the nematode feeds. The host organism or organism on which the nematode feeds may or may not be engineered to produce lower amounts of choline. For example, a host cell that does not naturally produce an inhibitor of a nematode PEAMT-like polypeptide can be transformed with genes encoding enzymes capable of making inhibitory analogs and provided with appropriate precursor chemicals exogenously if necessary. Alternatively, the active inhibitors and precursors can be made endogenously by the expression of the appropriate enzymes. In addition, yeast or other organisms can be modified to produce inhibitors. Nematodes that feed on such organisms would then be exposed to the inhibitors.

The ethanolamine analogs used in the invention can be applied to animals, plants or the environment of plants needing nematode control, or to the food of animals needing nematode control. The compositions may be applied by, for example drench or drip techniques. With drip applications ethanolamine analogs can be applied directly to the base of the plants or the soil immediately adjacent to the plants. The composition may be applied through existing drip irrigation systems. This procedure is particularly applicable for cotton, strawberries, tomatoes, potatoes, vegetables and ornamental plants. Alternatively, a drench application can be used where a sufficient quantity of nematicidal composition is applied such that it drains to the root area of the plants. The drench technique can be used for a variety of crops and turf grasses. The drench technique can also be used for animals. Preferably, the nematicidal compositions would be administered orally to promote activity against internal parasitic nematodes. Nematicidal compositions may also be administered in some cases by injection of the host animal.

The concentration of the nematicidal composition should be sufficient to control the nematode without causing phytotoxicity to the desired plant or undue toxicity to the animal host. An important aspect of the invention is the surprising discovery that certain ethanolamine analogs (e.g., N-ethylaniline, 4-(N-ethyl-N-methylamino)azobenzene, 2-(4-phenylazo-phenylamino)-ethanesulfonyl fluoride, 2-[4-(4-dimethylamino-phenylazo)-phenylamino]-ethanesulfonyl fluoride, 2-(4-phenylazo-phenylamino)-ethanesulfonamide, 2-[4-(4-dimethylamino-phenylazo)-phenylamino]-ethanesulfonamide, C,C,C-Trifluoro-N-(2-phenylamino-ethyl)-methanesulfonamide, 2-(diisopropylamino)ethanol, 2-(tert-butylamino)ethanol, N-(2-hydroxyethyl)aniline and D-phenylalaninol) that are predicted to be specific inhibitors of nematode PEAMTs are nematicidal. Thus, these analogs and their corresponding phosphate diesters, phosphonate diesters, fluorinated phosphonate diesters and sulfonate esters will provide useful compounds for nematode control.

The nematicidal ethanolamine analogs of the invention can be applied in conjunction with another nematicidal agent. The second agent may, for example, be applied simultaneously or sequentially. Such nematicidal agents can include for example, avermectins for animal applications.

A nematicidal ethanolamine analog may also be coupled to an agent such as glyphosate or polyoxyethylene sorbitan (Tween headgroup) to improve phloem mobility to the roots of plants.

The aforementioned nematicidal compositions can be used to treat diseases or infestations caused by nematodes of the following non-limiting, exemplary genera: Anguina, Ditylenchus, Tylenchorhynchus, Pratylenchus, Radopholus, Hirschmanniella, Nacobbus, Hoplolaimus, Scutellonema, Rotylenchus, Helicotylenchus, Rotylenchulus, Belonolaimus, Heterodera, other cyst nematodes, Meloidogyne, Criconemoides, Hemicycliophora, Paratylenchus, Tylenchulus, Aphelenchoides, Bursaphelenchus, Rhadinaphelenchus, Longidorus, Xiphinema, Trichodorus, and Paratrichodorus, Dirofiliaria, Onchocerca, Brugia, Acanthocheilonema, Aelurostrongylus, Anchlostoma, Angiostrongylus, Ascaris, Bunostomum, Capillaria, Chabertia, Cooperia, Crenosoma, Dictyocaulus, Dioctophyme, Dipetalonema, Dracunculus, Enterobius, Filaroides, Haemonchus, Lagochilascaris, Loa, Manseonella, Muellerius, Necator, Nematodirus, Oesophagostomum, Ostertagia, Parafilaria, Parascaris, Physaloptera, Protostrongylus, Setaria, Spirocerca, Stephanogilaria, Strongyloides, Strongylus, Thelazia, Toxascaris, Toxocara, Trichinella, Trichostrongylus, Trichuris, Uncinaria, and Wuchereria. Particularly preferred are nematodes including Dirofilaria, Onchocerca, Brugia, Acanthocheilonema, Dipetalonema, Loa, Mansonella, Parafilaria, Setaria, Stephanofilaria, and Wucheria, Pratylenchus, Heterodera, Meloidogyne, Paratylenchus. Species that are particularly preferred are: Ancylostoma caninum, Haemonchus contortus, Trichinella spiralis, Trichurs muris, Dirofilaria immitis, Dirofilaria tenuis, Dirofilaria repens, Dirofilari ursi, Ascaris suum, Toxocara canis, Toxocara cati, Strongyloides ratti, Parastrongyloides trichosuri, Heterodera glycines, Globodera pallida, Meloidogyne javanica, Meloidogyne incognita, and Meloidogyne arenaria, Radopholus similis, Longidorus elongatus, Meloidogyne hapla, and Pratylenchus penetrans.

The following examples are, therefore, to be construed as merely illustrative, and not limitative of the remainder of the disclosure in any way whatsoever. All of the publications cited herein are hereby incorporated by reference in their entirety.

### EXAMPLES

### Example 1

### RNA Mediated Interference (RNAi)

A double stranded RNA (dsRNA) molecule can be used to inactivate a phosphoethanolamine N-methyl transferase (PEAMT) gene in a cell by a process known as RNA mediated-interference (Fire et al. (1998) Nature 391:806-811, and Gönczy et al. (2000) Nature 408:331-336). The dsRNA molecule can have the nucleotide sequence of a PEAMT nucleic acid (preferably exonic) or a fragment thereof. For example, the molecule can comprise at least 50, at least 100, at least 200, at least 300, or at least 500 or more contiguous nucleotides of a PEAMT-like gene. The dsRNA molecule can be delivered to nematodes via direct injection, by soaking nematodes in aqueous solution containing concentrated dsRNA, or by raising bacteriovorous nematodes on E. coli genetically engineered to produce the dsRNA molecule (Kamath et al. (2000) Genome Biol. 2; Tabara et al. (1998) Science 282:430-431).

### PEAMT RNAi by Feeding:

C. elegans can be grown on lawns of E. coli genetically engineered to produce double-stranded RNA (dsRNA) designed to inhibit PEAMT1 or PEAMT2 expression. Briefly, E. coli were transformed with genomic fragments encoding portions of the C. elegans PEAMT1 or the PEAMT2 gene. Specifically, a 960 nucleotide fragment was amplified from the PEAMT1 gene using oligo-nucleotide primers containing the sequences 5′-ATGGTGAACGTTCGTCGTGC-3′ and 5′-CATACGTATTTCTCATCATC-3′ respectively, or an 854 nucleotide fragment was amplified from the PEAMT2 gene using oligo-nucleotide primers containing the sequences 5′-CCAGATTATTACCAACGCCG-3′ and 5′-TGAACTTACATAGATTCTTG-3′ respectively. The PEAMT1 and PEAMT2 genomic fragments were cloned separately into an E. coli expression vector between opposing T7 polymerase promoters. The clone was then transformed into a strain of E. coli that carries an IPTG-inducible T7 polymerase. As a control, E. coli was transformed with a gene encoding the Green Fluorescent Protein (GFP). Feeding RNAi was initiated from C. elegans larvae at 23° C. on NGM plates containing IPTG and E. coli expressing the C. elegans PEAMT1 or PEAMT2, or GFP dsRNA. If the starting worm (the P0) was an L1, or a dauer larva, the phenotype of both the PEAMT1 and PEAMT2 RNAi-generated mutants was complete or almost complete sterility. One the other hand, if the P0 animal was an L4 larva, then the phenotype of both the PEAMT1 and PEAMT2 RNAi-generated mutants was L1/L2 larval arrested development and lethality. The sequence of the PEAMT1 and PEAMT2 genes is of sufficiently high complexity (i.e., unique) such that the RNAi is not likely to represent cross reactivity with other genes.

C. elegans cultures grown in the presence of E. coli expressing dsRNA from the PEAMT1 or the PEAMT2 gene were strongly impaired indicating that the PEAMT genes provide essential functions in nematodes and that dsRNA from the PEAMT-like genes is lethal when ingested by C. elegans. These results demonstrate that PEAMT's are important for the viability of C. elegans and suggest that they are useful targets for the development of compounds that reduce the viability of nematodes.

### Example 2

### Chemical Rescue of the PEAMT 1 and PEAMT2 RNAi-Generated Phenotype

The experiments described below were designed to test whether the PEAMT1/PEAMT2 RNAi knockout phenotype can be rescued by providing C. elegans with the products downstream of the predicted PEAMT reaction catalyzed by the enzymes. The free bases (EA, MME, DME and Cho) were added to the bacterial medium and it was assumed that these would be taken up and converted to the corresponding phosphobases by the actions of ethanolamine/choline kinases.

C. elegans worms were fed bacteria expressing dsRNA homologous to PEAMT1, PEAMT2, actin, or GFP along with specific chemicals (EA, MME, DME or Cho). Chemicals were added to NGM plates at various concentrations and negative (GFP dsRNA) and positive (actin dsRNA) controls were performed for each chemical or chemical mixture at each concentration. Specifically, agar plates containing NGM and the chemicals specified in Table 1 (see below) were seeded with bacteria expressing double-stranded RNA homologous to either PEAMT1 or PEAMT2. In some experiments a single L1 or dauer larva was placed on each plate, and the P0 and the F1 were examined for the next 5 days. In other experiments, a single L4 C. elegans hermaphrodite was placed on each plate. The hermaphrodite was allowed to lay eggs for 24 hours and the phenotype of the F1 progeny was scored 48 hours after the initial 24-hour egg-laying period. At the time of scoring, 4 individual F1 progeny were cloned to separate plates containing the same chemical and bacteria they were grown on. The F1 and F2 progeny were examined over the next 4-5 days for the presence of a phenotype.

TABLE 1
^{C. elegans} PEAMT1 and PEAMT2 RNAi feeding phenotypes (starting
with C. elegans L1, dauer, or L4 larva as the P0 animal).

| | Compounds added to | F1 phenotype | |
|---|---|---|---|
| P0 | the plate media | PEAMT1 dsRNA | PEAMT2 dsRNA |
| L1 | None | Sterility | Sterility |
| | 10 mM DME | Fertile adults | Sterility |
| Dauer | None | Partial sterility | Partial sterility |
| | 10 mM DME | Fertile adults | Sterility |
| L4 | None | L1/L2 arrest/lethality | L1/L2 arrest/lethality |
| | 10 mM ethanolamine (EA) | L1/L2 arrest/lethality | L1/L2 arrest/lethality |
| | 5 or 10 mM MME | Fertile adults | L1/L2 arrest/lethality |
| | 5 or 10 mM DME | Fertile adults | L1/L2 arrest/lethality |
| | 5 mM choline (Cho) | L1/L2 arrest/lethality | L1/L2 arrest/lethality |
| | 10 or 15 mM Cho | Sterile adults | L1/L2 arrest/lethality |
| | 25 mM or 30 mM Cho | Fertile adults | Fertile adults |
| | 5 mM each EA, MME | Fertile adults | L1/L2 arrest/lethality |
| | 5 mM each EA, DME | | |
| | 5 mM each EA, Cho | | |
| | 5 mM each MME, DME | | |
| | 5 mM each MME, Cho | | |
| | 5 mM each DME, Cho | | |
| | 5 mM each MME, DME, Cho | | |

The C. elegans phosphoethanolamine N-methyltransferase proteins PEAMT1 and PEAMT2 together catalyze the conversion of phosphoethanolamine to phosphocholine. The RNAi-generated mutants of PEAMT1 or PEAMT2 are both predicted to have decreased levels of choline which leads to sterility, or L1/L2 larval arrested development and death. Addition of 25 mM choline rescues the larval arrest associated with both PEAMT1 and PEAMT2 RNAi phenotypes. However, only the PEAMT1 mutants are rescued by the addition of 5 mM monoethanolamine (MME) or 5 mM dimethylethanolamine (DME) while the PEAMT2 mutants are not (see Table 1). These data are consistent with the prediction that PEAMT1 catalyzes the first methylation while PEAMT2 catalyzes the second and third methylations in the conversion of pEA to pCho:

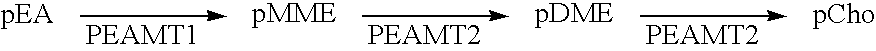

Five mM DME rescues the sterility associated with PEAMT1 RNAi. The rescue by DME strongly suggests the sterility is due to a reduction in choline production and not due to other changes caused by the PEAMT mutations.

The data also demonstrate that when choline alone is used as the rescuing chemical, 25 mM choline is required to complement the PEAMT1 and PEAMT2 RNAi phenotypes. This suggests that chemicals that interfere with this pathway will not likely be counteracted by the amount of choline nematodes can acquire from the environment.

### Example 3

### Nematicidal Activity of Small Molecules Structurally Similar to Ethanolamine Against Caenorhabditis Elegans

The structures of ethanolamine-like molecules tested against C. elegans for nematicidal activity are shown below.

TABLE 2

| COMPOUND | STRUCTURE |
|---|---|
| 2-(diisopropylamino)ethanol (N-substituted) | 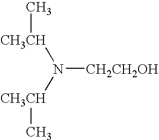 |
| 2-(tert-butylamino)ethanol (N-substituted) | 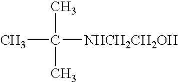 |
| D-phenylalaninol (C2-substituted) | 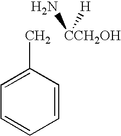 |
| 2-amino-1-phenylethanol (C1-substituted) | 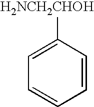 |
| N-(2-hydroxyethyl)aniline (N-substituted) | 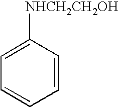 |

One approach to the development of chemicals that interfere with the function of an enzyme is to identify compounds that mimic substrate binding but that cannot be acted on by the enzyme. Therefore, several ethanolamine-derived compounds were tested for the ability to kill C. elegans in culture. Compounds with substitutions at various positions on ethanolamine were tested including some with substitutions on the nitrogen, the carbon adjacent to the nitrogen (C2), and on the carbon adjacent to the oxygen (C1).

A single C. elegans L4 larva (the P0 animal) was placed on a lawn of E. coli that had been spotted onto NGM plates containing various concentrations of the ethanolamine-like compounds. The growth and development of the P0 and its F1 progeny at 23° C. was monitored by visual observation over several days. Four of the compounds tested [2-(diisopropylamino)ethanol, 2-(tert-butylamino)ethanol, D-phenylalaninol and N-(2-hydroxyethyl)aniline], showed nematicidal activity against C. elegans. In addition, the phenotype of worms treated with the nematicidal ethanolamine-like compounds mimicked the RNAi-phenotype of PEAMT1 and PEAMT2. That is, the F1 progeny of the treated worm did not develop beyond the L1/L2 stage and died. Treatment of C. elegans with the C1-substituted compound 2-amino-1-phenylethanol showed no nematicidal effect.

TABLE 3
Nematicidal activity of ethanolamine-like
compounds against C. elegans.

| | CONCEN- | |
|---|---|---|
| COMPOUND | TRATION | F1 PHENOTYPE |
| 2-(diisopropylamino)ethanol | 10 mM | L1/L2 arrest/lethality |
| 2-(tert-butylamino)ethanol | 10 mM | L1/L2 arrest/lethality |
| D-phenylalaninol | 10 mM | L1/L2 arrest/lethality |
| 2-amino-1-phenylethanol | 25 mM | Wild-type development |
| N-(2-hydroxyethyl)aniline | 10 mM | L1/L2 arrest/lethality |
| Control (no compound) | Not ap- | Wild-type development |
| | plicable | |

### Example 4

TABLE 4
Nematicidal activity of ethanolamine-like compounds against other nematodes.

| COMPOUND | SPECIES | CONCENTRATION | | F1 PHENOTYPE |
|---|---|---|---|---|
| diisopropylamino)ethanol | A. ellesmerensis | 10 | mM | L1/L2 arrest/lethality |
| | Cephalobus sp. | 10 | mM | L1/L2 arrest/lethality |
| 2-(tert-butylamino)ethanol | A. ellesmerensis | 10 | mM | L1/L2 arrest/lethality |
| | Cephalobus sp. | 10 | mM | L1/L2 arrest/lethality |
| D-phenylalaninol | A. ellesmerensis | 12.5 | mM | L1/L2 arrest/lethality |
| | Cephalobus sp. | 12.5 | mM | L1/L2 arrest/lethality |

| | | | |
|---|---|---|---|
| Control (no compound) | Cephalobus sp. | not applicable | Wild-type |

The ethanolamine-like compounds mentioned above are also nematicidal against Acrobiloides ellesmerensis and Cephalobus sp. Assays were done as those described for C. elegans L4 larvae. Three of the four compounds that were nematicidal against C. elegans were tested and were found to be nematicidal against A. ellesmerensis and Cephalobus sp.

Sulfonic, phosphonic, or phosphate prodrugs based on the structures of the molecules discussed here will provide better activity than the parent molecules themselves. Enzymes like PEAMT1 and PEAMT2, which interact with phosphorylated substrates, bind more tightly to the phosphorylated forms of the substrate than to the non-phosphorylated forms. For example, in the case of SH2 domains, phosphorylated peptides exhibit binding four orders of magnitude greater than non-phosphorylated peptides (Bradshaw et al, (1999) J. Mol. Biol. 293(4):971-85). Therefore, the addition of a phosphate, or a phosphate mimic to the ethanolamine-like compounds will increase the affinity for the enzyme making them more potent inhibitors of the PEAMT enzymes.

### Example 5

TABLE 5
Nematicidal activity of a variety of ethanolamine-
like compounds against C. elegans.

| | CHEMICAL | EC₅₀ |
|---|---|---|
| COMPOUND | NAME | (mM) |
| 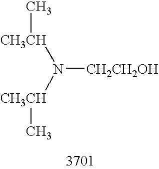 | 2-(Diisopropylamino)ethanol | 4.7 |
| 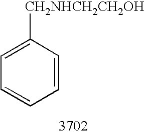 | 2-Benzylaminoethanol | 3.4 |
| 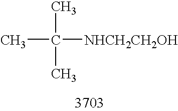 | 2-(tert-Butylamino)ethanol | 4.1 |
| 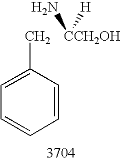 | D-Phenylalaninol | 2.5 |
| 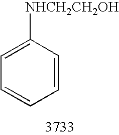 | N-(2-Hydroxyethyl)aniline | 4.2 |
| 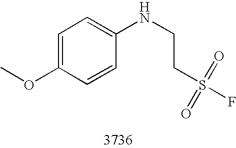 | 2-4-methoxy-phenylamino- ethanesulfonyl fluoride | 0.5 |
| 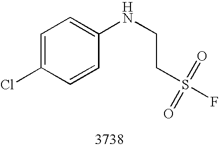 | 2-4-chlorophenylaminoethane- sulfonyl fluoride | 0.082 |
| 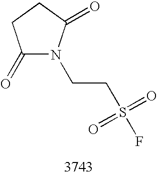 | 2,5-Dioxo-1-pyrrolidineethane- sulfonyl fluoride | 1.3 |
| 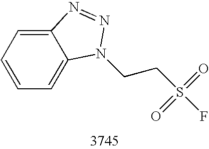 | 2-Benzotriazol-1-yl-ethanesulfonylfluoride | 1.7 1.34 |
| 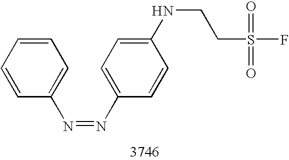 | 2-(4-phenylazo-phenylamino)- ethanesulfonyl fluoride | 0.002 |
| 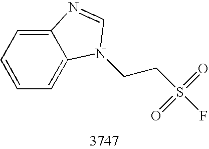 | 2-Benzoimidazol-1-yl- ethanesulfonyl fluoride | 1.4 |
| 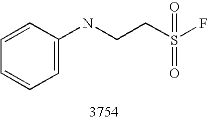 | 2-phenylaminoethanesulfonyl fluoride | 0.94 0.57 |
| 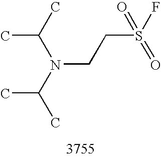 | 2-diisopropylaminoethanesulfonyl fluoride | 0.75 |
| 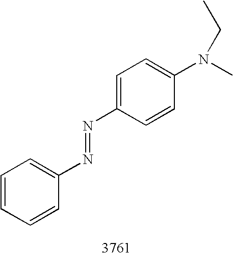 | 4-N-ethyl-N-methylaminoazobenzene | 0.007 |
| 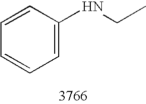 | N-ethylaniline | 0.36 |
| 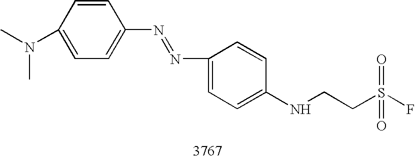 | 2-[4-(4-dimethylamino-phenylazo)- phenylamino]-ethanesulfonyl fluoride | 0.007 |
| 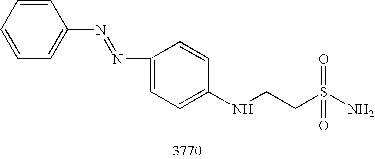 | 2-[(4-phenylazo)phenylanilino] ethanesulfonamide | 0.02 |

EC50's of compounds against C. elegans were measured in a contact assay. Compounds were solubilized in acetone, ethanol or water (in that order of preference) at 100× the desired concentration. Dilution series of 10×, 3×, 2× or square root-2× were accomplished by serial dilution with identical solvent. Between 6 and 12 concentration points were assayed. For each concentration, 50 microliters of 100× compound solution were added to 5 ml NGM-agar at 50 to 60° C. Four wells of a 24-well plate each received approximately 1 ml of the NGM-agar-compound mixture. Following overnight cooling, 8 microlitres of a fresh culture of OP50 bacteria was added to each well, and this was incubated overnight at room temperature. One L4 stage C. elegans hermaphrodite worm (strain N2) was added to each well. Plates were incubated at 20° C. At 96 hours after worm addition, each well was scored for number of adults, number of eggs and number of larvae present, as well as for presence or absence of crystallized compound, cloudiness of plates, and depletion of bacterial food source. Most plates were also scored at 120 or 144 hours following challenge. For determination of an EC50, the average number of adults present in the 4 replicate wells 96 hours after challenge was determined, and an EC50 interpolated.

### Example 6

### Greenhouse Assay of 2-(4-Phenylazo-Phenylamino)-Ethanesulfonyl Fluoride (3746) and Preliminary Assessment of Non-Target Effects

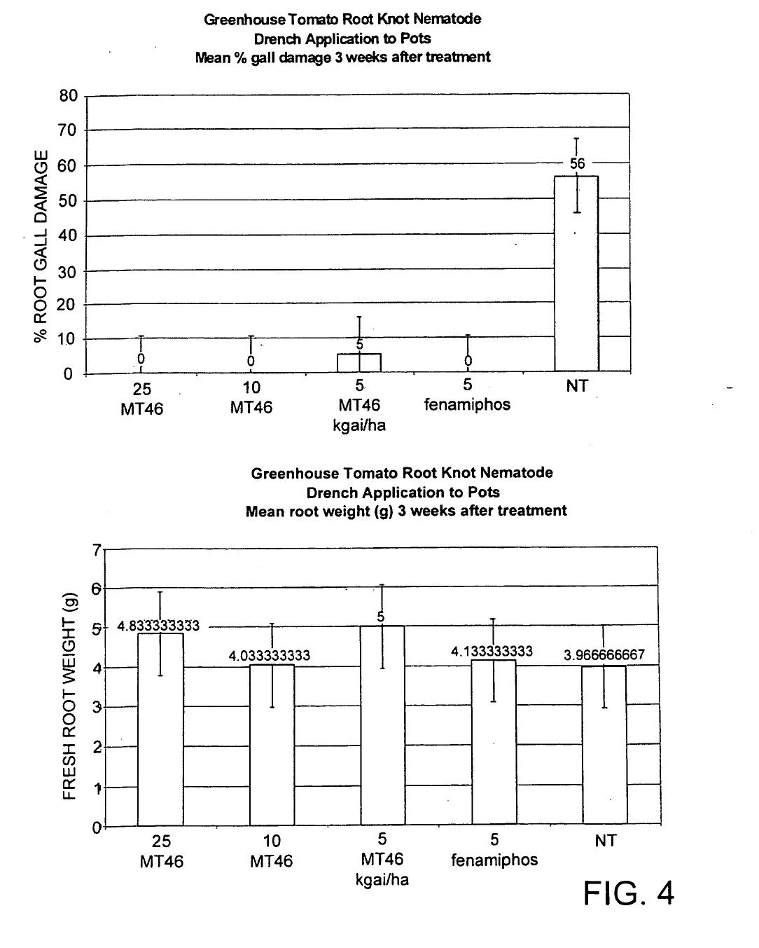

As seen in FIG. 4, 2-(4-phenylazo-phenylamino)-ethanesulfonyl fluoride shows nematode control approaching that of the commercial nematicides fenamiphos (Nemacur) in drench (soil based) assays against root knot nematode infections of tomato plants in the greenhouse. Furthermore, 3746 shows no phytoxicity at any of the rates tested. Additionally, as is seen in the table 6 below 2-(4-phenylazo-phenylamino)-ethanesulfonyl fluoride is not toxic to several arthropods. Low to moderate toxicity is seen with various fungal species. The lack of general (i.e., non-specific) toxicity of 3746 is consistent with the killing of C. elegans in vitro and control of M. incognita infection in tomato pot assays being due to inhibition of essential nematode phosphoethanolamine n-methyltransferases.

TABLE 6
Fungal and arthropod toxicity of 3746

| | | Concentration | |
|---|---|---|---|
| Organism | | (μM) | Result |
| Fungi | Sclerotinia sclerotiorum | 163 | >75% growth inhibition |
| | Sclerotinia sclerotiorum | 16.3 | <25% growth inhibition |
| | Fusarium graminearum | 16.3 | >75% growth inhibition |
| | Fusarium graminearum | 1.63 | <25% growth inhibition |
| | Alternaria solani | 16.3 | >75% growth inhibition |
| | Alternaria solani | 1.63 | <25% growth inhibition |
| | Botrytis cinerea | 16.3 | >75% growth inhibition |
| | Botrytis cinerea | 1.63 | <25% growth inhibition |
| Arthropod | Beet army worm | 25000 | Lethal |
| | Beet army worm | 2500 | No effect |
| | Corn ear worm | 25000 | Lethal |
| | Corn ear worm | 2500 | Non-effect |

1. A nematicidal composition, comprising: an effective amount of a compound or a mixture of compounds having the formula: 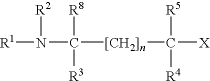
wherein:
each R¹, R², R³ and R⁸ is, independently, singly or multiply substituted or unsubstituted alkyl, cycloalkyl, alkenyl, alkynyl, aryl or heteroaryl group or a hydroxy, alkoxy, oxo, or hydrogen;
each R⁴ and R⁵ is, independently, hydrogen or halogen;
n=0 or 1;
X is H or an alkyl group or —OH or —OPO₃H₂ (phosphate), or —PO₃H₂ (phosphonate), or —SO₃H (sulfonate), or —SO₂F (sulfonyl fluoride), —SO₂NH₂ (sulfonamide), —NHSO₂CF₃ (trifluoromethyl sulfonamide), —SO₂CF₃ (trifluoromethyl sulfone), —OPO₃R⁶R⁷ (phosphate ester or diester), —PO₃R⁶R⁷ (phosphonate ester or diester), —SO₃R⁶ (sulfonate ester);
R⁶ is an unsubstituted, singly substituted or multiply substituted alkyl, phenyl, alkoxyalkyl or alkoxyaryl;
R⁷ is an unsubstituted, singly substituted or multiply substituted alkyl, phenyl, alkoxyalkyl or alkoxyaryl.

2-90. (canceled)
